(19) United States
(12) Patent Application Publication
(10) Pub. No.: US 2007/0003628 A1
(43) Pub. Date: Jan. 4, 2007

(54) Nanoparticulate clopidogrel formulations

(75) Inventors: Gary G. Liversidge, West Chester, PA (US); Scott Jenkins, Downingtown, PA (US)

Correspondence Address: ELAN DRUG DELIVERY, INC.;C/O FOLEY & LARDNER LLP, 3000 K STREET, N.W., SUITE 500, WASHINGTON, DC, 20007-5109, US

(73) Assignee: Elan Pharma International Limited

(21) Appl. No.: 11/430,180

(22) Filed: May 9, 2006

Related U.S. Application Data
(60) Provisional application No. 60/679,398, filed on May 10, 2005.

(51) Int. Cl. A61K 9/14

(52) U.S. Cl. 424/489; 977/906

(57) ABSTRACT

The present invention is directed to compositions comprising a nanoparticulate clopidogrel, or a salt or derivative thereof, having improved bioavailability. The nanoparticulate clopidogrel particles of the composition have an effective average particle size of less than about 2000 nm and are useful in the prevention and treatment of pathologies induced by platelet aggregation. The clopidogrel particles may also be formulated as a controlled release polymeric coating or matrix drug delivery system.

### CROSS REFERENCE TO RELATED APPLICATIONS

This application claims priority to U.S. Provisional Patent Application Ser. No. 60/679,398, filed on May 9, 2005.

### FIELD OF INVENTION

The present invention relates generally to compounds and compositions useful in the prevention and treatment of pathological states induced by platelet aggregation. More specifically, the invention relates to nanoparticulate clopidogrel, or a salt or derivative thereof, and compositions comprising the same. The nanoparticulate clopidogrel compositions may have an effective average particle size of less than about 2000 nm. The invention also relates to methods of making and using nanoparticulate clopidogrel compositions.

### BACKGROUND

A. Background Regarding Clopidogrel

With the exception of the year 1918, cardiovascular disease has been the number one killer in the United States every year since 1900. Heart Disease and Stroke Statistics—2006 Update: A Report from the American Heart Association Statistics Committee and Stroke Statistics Subcommittee, Circulation Feb. 14, 2006. Every day, nearly 2500 Americans die of cardiovascular and related disease. This is more than the next four leading causes of death combined (cancer, chronic lower respiratory diseases, accidents and diabetes mellitus). Id. Examples of cardiovascular and related diseases include various types of strokes, (e.g., embolic stroke, ischemic stroke, and transient ischemic stroke), peripheral artery disease, blood clots (e.g., thrombus or embolism), and coronary artery disease, which can lead to myocardial infarction, angina pectoris, and heart failure.

Both heart attacks and strokes can be caused by blood clots that occlude an artery, such as a coronary artery in the case of heart attack, or an artery leading to the brain or an artery in the brain in the case of stroke. Clots may form for a variety of reasons—a common cause, however, is atherosclerosis. In atherosclerosis, fat and cholesterol build up inside an artery, hardening the arterial wall and narrowing the arterial passage. This atherosclerotic buildup occasionally breaks free or cracks, triggering clot formation which may lead to cardiovascular trauma. Clots may also form around the atherosclerotic plaque deposits.

Preventative measures and treatments common to such conditions include therapies that prevent platelet aggregation. For example, anti-coagulant therapies including warfarin and heparin target key factors in the clotting cascade such as Factor II, VII, IX and X, while anti-platelet therapies such as aspirin inhibit platelet clumping or aggregation during clot formation. Aspirin works by preventing the formation of thromboxane, a key clotting factor produced by platelets.

Another anti-platelet drug, clopidogrel, inhibits ADP-induced platelet aggregation by direct inhibition of adenosine diphosphate (ADP) binding to its receptor and of the subsequent ADP-mediated activation of the glycoprotein GPIIb/IIa complex. This also inhibits platelet aggregation induced by agonists other than ADP by blocking the amplification of platelet activation by released ADP.

The chemical name for clopidogrel bisulfate is methyl (+)-(S)-α-(2-chorophenyl)-6,7-dihydrothieno[3,2-c]pyridine-5(4H)-acetate sulfate (1:1). The empirical formula of clopidogrel bisulfate is C₁₆H₁₆Cl NO₂S.H₂SO₄ and its molecular weight is 419.9. The structural formula is as follows:

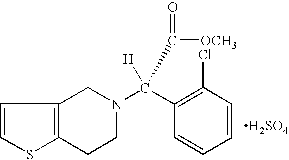

Clopidogrel bisulfate is a white to off-white powder. It is practically insoluble in water at neutral pH but is freely soluble at pH 1.0. It also dissolves freely in methanol, it dissolves sparingly in methylene chloride, and is practically insoluble in ethyl ether.

Clopidogrel bisulfate is commercially available under the registered trademark PLAVIX® by Bristol-Myers Squibb/Sanofi Pharmaceuticals Parternship of New York, N.Y. PLAVIX® is administered as an oral tablet at a recommended dose of 75 mg once daily. PLAVIX® is provided as pink, round, biconvex, debossed film-coated tablets containing 97.875 mg of clopidogrel bisulfate which is the molar equivalent of 75 mg of clopidogrel base.

Clopidogrel bisulfate is indicated for the reduction of thrombotic events such as recent myocardial infarction (MI), recent stroke or established arterial disease, and has been shown to reduce the rate of a combined end point of new ischemic stroke, new MI, and other vascular death. For patients with acute coronary syndrome, clopidogrel bisulfate has been shown to decrease the rate of a combined end point of cardiovascular death, MI, or stroke as well as the rate of a combined end point of cardiovascular death, MI, stroke, or refractory ischemia.

Clopidogrel has been described, for example, in U.S. Pat. No. 4,847,265 for “Dextro-Rotatory Enantiomer of Methyl Alpha-5 (4,5,6,7-Tetrahydro (3,2-c) Thieno Pyridyl) (2-Chlorophenyl)-Acetate and the Pharmaceutical Compositions Containing It”, U.S. Pat. No. 5,576,328 for “Method for the Secondary Prevention of Ischemic Events”, U.S. Pat. No. 5,989,578 for “Associations of Active Principles Containing Clopidogrel and an Anti-thrombotic Agent”, U.S. Pat. Nos. 6,429,210 and 6,504,030 both for “Polymorphic Clopidogrel Hydrogen Sulphate Form”, U.S. Pat. No. 6,635,763 for “Process to Prepare Clopidogrel”, U.S. Pat. Nos. 6,737,411 and 6,800,759 both for “Racemization and Enantiomer Separation of Clopidogrel”, and U.S. Pat. No. 6,858,734 for “Preparation of (S)-Clopidogrel and Related Compounds”.

Clopidogrel has high therapeutic value in the prevention and treatment of pathologies induced by platelet aggregation. However, because clopidogrel is practically insoluble in water, significant bioavailability can be problematic. There is a need in the art for nanoparticulate clopidogrel formulations which overcome this and other problems associated with the use of clopidogrel in the prevention and treatment of pathologies induced by platelet aggregation. The present invention satisfies this need.

The present invention then, relates to a nanoparticulate clopidogrel, or a salt or derivative thereof, composition for the treatment of cardiovascular disease. Moreover, the present invention further comprises nanoparticulate clopidogrel particles that have been coated with one or more polymeric coatings for a sustained and/or delayed controlled drug release.

B. Background Regarding Nanoparticulate Active Agent Compositions

Nanoparticulate active agent compositions, first described in U.S. Pat. No. 5,145,684 (“the '684 patent”), are particles consisting of a poorly soluble therapeutic or diagnostic agent having adsorbed onto the surface thereof a non-crosslinked surface stabilizer. The '684 patent does not describe nanoparticulate compositions of clopidogrel.

Methods of making nanoparticulate active agent compositions are described in, for example, U.S. Pat. Nos. 5,518,187 and 5,862,999, both for “Method of Grinding Pharmaceutical Substances;” U.S. Pat. No. 5,718,388, for “Continuous Method of Grinding Pharmaceutical Substances;” and U.S. Pat. No. 5,510,118 for “Process of Preparing Therapeutic Compositions Containing Nanoparticles.”

Nanoparticulate compositions are also described, for example, in U.S. Pat. No. 5,298,262 for “Use of Ionic Cloud Point Modifiers to Prevent Particle Aggregation During Sterilization;” U.S. Pat. No. 5,302,401 for “Method to Reduce Particle Size Growth During Lyophilization;” U.S. Pat. No. 5,318,767 for “X-Ray Contrast Compositions Useful in Medical Imaging;” U.S. Pat. No. 5,326,552 for “Novel Formulation For Nanoparticulate X-Ray Blood Pool Contrast Agents Using High Molecular Weight Non-ionic Surfactants;” U.S. Pat. No. 5,328,404 for “Method of X-Ray Imaging Using iodinated Aromatic Propanedioates;” U.S. Pat. No. 5,336,507 for “Use of Charged Phospholipids to Reduce Nanoparticle Aggregation;” U.S. Pat. No. 5,340,564 for “Formulations Comprising Olin 10-G to Prevent Particle Aggregation and Increase Stability;” U.S. Pat. No. 5,346,702 for “Use of Non-Ionic Cloud Point Modifiers to Minimize Nanoparticulate Aggregation During Sterilization;” U.S. Pat. No. 5,349,957 for “Preparation and Magnetic Properties of Very Small Magnetic-Dextran Particles;” U.S. Pat. No. 5,352,459 for “Use of Purified Surface Modifiers to Prevent Particle Aggregation During Sterilization;” U.S. Pat. Nos. 5,399,363 and 5,494,683, both for “Surface Modified Anticancer Nanoparticles;” U.S. Pat. No. 5,401,492 for “Water Insoluble Non-Magnetic Manganese Particles as Magnetic Resonance Enhancement Agents;” U.S. Pat. No. 5,429,824 for “Use of Tyloxapol as a Nanoparticulate Stabilizer;” U.S. Pat. No. 5,447,710 for “Method for Making Nanoparticulate X-Ray Blood Pool Contrast Agents Using High Molecular Weight Non-ionic Surfactants;” U.S. Pat. No. 5,451,393 for “X-Ray Contrast Compositions Useful in Medical Imaging;” U.S. Pat. No. 5,466,440 for “Formulations of Oral Gastrointestinal Diagnostic X-Ray Contrast Agents in Combination with Pharmaceutically Acceptable Clays;” U.S. Pat. No. 5,470,583 for “Method of Preparing Nanoparticle Compositions Containing Charged Phospholipids to Reduce Aggregation;” U.S. Pat. No. 5,472,683 for “Nanoparticulate Diagnostic Mixed Carbamic Anhydrides as X-Ray Contrast Agents for Blood Pool and Lymphatic System Imaging;” U.S. Pat. No. 5,500,204 for “Nanoparticulate Diagnostic Dimers as X-Ray Contrast Agents for Blood Pool and Lymphatic System Imaging;” U.S. Pat. No. 5,518,738 for “Nanoparticulate NSAID Formulations;” U.S. Pat. No. 5,521,218 for “Nanoparticulate Iododipamide Derivatives for Use as X-Ray Contrast Agents;” U.S. Pat. No. 5,525,328 for “Nanoparticulate Diagnostic Diatrizoxy Ester X-Ray Contrast Agents for Blood Pool and Lymphatic System Imaging;” U.S. Pat. No. 5,543,133 for “Process of Preparing X-Ray Contrast Compositions Containing Nanoparticles;” U.S. Pat. No. 5,552,160 for “Surface Modified NSAID Nanoparticles;” U.S. Pat. No. 5,560,931 for “Formulations of Compounds as Nanoparticulate Dispersions in Digestible Oils or Fatty Acids;” U.S. Pat. No. 5,565,188 for “Polyalkylene Block Copolymers as Surface Modifiers for Nanoparticles;” U.S. Pat. No. 5,569,448 for “Sulfated Non-ionic Block Copolymer Surfactant as Stabilizer Coatings for Nanoparticle Compositions;” U.S. Pat. No. 5,571,536 for “Formulations of Compounds as Nanoparticulate Dispersions in Digestible Oils or Fatty Acids;” U.S. Pat. No. 5,573,749 for “Nanoparticulate Diagnostic Mixed Carboxylic Anydrides as X-Ray Contrast Agents for Blood Pool and Lymphatic System Imaging;” U.S. Pat. No. 5,573,750 for “Diagnostic Imaging X-Ray Contrast Agents;” U.S. Pat. No. 5,573,783 for “Redispersible Nanoparticulate Film Matrices With Protective Overcoats;” U.S. Pat. No. 5,580,579 for “Site-specific Adhesion Within the GI Tract Using Nanoparticles Stabilized by High Molecular Weight, Linear Poly(ethylene Oxide) Polymers;” U.S. Pat. No. 5,585,108 for “Formulations of Oral Gastrointestinal Therapeutic Agents in Combination with Pharmaceutically Acceptable Clays;” U.S. Pat. No. 5,587,143 for “Butylene Oxide-Ethylene Oxide Block Copolymers Surfactants as Stabilizer Coatings for Nanoparticulate Compositions;” U.S. Pat. No. 5,591,456 for “Milled Naproxen with Hydroxypropyl Cellulose as Dispersion Stabilizer;” U.S. Pat. No. 5,593,657 for “Novel Barium Salt Formulations Stabilized by Non-ionic and Anionic Stabilizers;” U.S. Pat. No. 5,622,938 for “Sugar Based Surfactant for Nanocrystals;” U.S. Pat. No. 5,628,981 for “Improved Formulations of Oral Gastrointestinal Diagnostic X-Ray Contrast Agents and Oral Gastrointestinal Therapeutic Agents;” U.S. Pat. No. 5,643,552 for “Nanoparticulate Diagnostic Mixed Carbonic Anhydrides as X-Ray Contrast Agents for Blood Pool and Lymphatic System Imaging;” U.S. Pat. No. 5,718,388 for “Continuous Method of Grinding Pharmaceutical Substances;” U.S. Pat. No. 5,718,919 for “Nanoparticles Containing the R(−)Enantiomer of Ibuprofen;” U.S. Pat. No. 5,747,001 for “Aerosols Containing Beclomethasone Nanoparticle Dispersions;” U.S. Pat. No. 5,834,025 for “Reduction of Intravenously Administered Nanoparticulate Formulation Induced Adverse Physiological Reactions;” U.S. Pat. No. 6,045,829 “Nanocrystalline Formulations of Human Immunodeficiency Virus (HIV) Protease Inhibitors Using Cellulosic Surface Stabilizers;” U.S. Pat. No. 6,068,858 for “Methods of Making Nanocrystalline Formulations of Human Immunodeficiency Virus (HIV) Protease Inhibitors Using Cellulosic Surface Stabilizers;” U.S. Pat. No. 6,153,225 for “Injectable Formulations of Nanoparticulate Naproxen;” U.S. Pat. No. 6,165,506 for “New Solid Dose Form of Nanoparticulate Naproxen;” U.S. Pat. No. 6,221,400 for “Methods of Treating Mammals Using Nanocrystalline Formulations of Human Immunodeficiency Virus (HIV) Protease Inhibitors;” U.S. Pat. No. 6,264,922 for “Nebulized Aerosols Containing Nanoparticle Dispersions;” U.S. Pat. No. 6,267,989 for “Methods for Preventing Crystal Growth and Particle Aggregation in Nanoparticle Compositions;” U.S. Pat. No. 6,270,806 for “Use of PEG-Derivatized Lipids as Surface Stabilizers for Nanoparticulate Compositions;” U.S. Pat. No. 6,316,029 for “Rapidly Disintegrating Solid Oral Dosage Form,” U.S. Pat. No. 6,375,986 for “Solid Dose Nanoparticulate Compositions Comprising a Synergistic Combination of a Polymeric Surface Stabilizer and Dioctyl Sodium Sulfosuccinate;” U.S. Pat. No. 6,428,814 for “Bioadhesive Nanoparticulate Compositions Having Cationic Surface Stabilizers;” U.S. Pat. No. 6,431,478 for “Small Scale Mill;” and U.S. Pat. No. 6,432,381 for “Methods for Targeting Drug Delivery to the Upper and/or Lower Gastrointestinal Tract,” U.S. Pat. No. 6,592,903 for “Nanoparticulate Dispersions Comprising a Synergistic Combination of a Polymeric Surface Stabilizer and Dioctyl Sodium Sulfosuccinate,” U.S. Pat. No. 6,582,285 for “Apparatus for sanitary wet milling;” U.S. Pat. No. 6,656,504 for “Nanoparticulate Compositions Comprising Amorphous Cyclosporine;” U.S. Pat. No. 6,742,734 for “System and Method for Milling Materials;” U.S. Pat. No. 6,745,962 for “Small Scale Mill and Method Thereof;” U.S. Pat. No. 6,811,767 for “Liquid droplet aerosols of nanoparticulate drugs;” and U.S. Pat. No. 6,908,626 for “Compositions having a combination of immediate release and controlled release characteristics;” U.S. Pat. No. 6,969,529 for “Nanoparticulate compositions comprising copolymers of vinyl pyrrolidone and vinyl acetate as surface stabilizers;” U.S. Pat. No. 6,976,647 for “System and Method for Milling Materials,” all of which are specifically incorporated by reference. In addition, U.S. Patent Publication No. 20020012675 A1, for “Controlled Release Nanoparticulate Compositions;” U.S. Patent Publication No. 20050276974 for “Nanoparticulate Fibrate Formulations;” U.S. Patent Publication No. 20050238725 for “Nanoparticulate compositions having a peptide as a surface stabilizer;” U.S. Patent Publication No. 20050233001 for “Nanoparticulate megestrol formulations;” U.S. Patent Publication No. 20050147664 for “Compositions comprising antibodies and methods of using the same for targeting nanoparticulate active agent delivery;” U.S. Patent Publication No. 20050063913 for “Novel metaxalone compositions;” U.S. Patent Publication No. 20050042177 for “Novel compositions of sildenafil free base;” U.S. Patent Publication No. 20050031691 for “Gel stabilized nanoparticulate active agent compositions;” U.S. Patent Publication No. 20050019412 for” Novel glipizide compositions;” U.S. Patent Publication No. 20050004049 for “Novel griseofulvin compositions;” U.S. Patent Publication No. 20040258758 for “Nanoparticulate topiramate formulations;” U.S. Patent Publication No. 20040258757 for” Liquid dosage compositions of stable nanoparticulate active agents;” U.S. Patent Publication No. 20040229038 for “Nanoparticulate meloxicam formulations;” U.S. Patent Publication No. 20040208833 for “Novel fluticasone formulations;” U.S. Patent Publication No. 20040195413 for” Compositions and method for milling materials;” U.S. Patent Publication No. 20040156895 for “Solid dosage forms comprising pullulan;” U.S. Patent Publication No. U.S. Patent Publication No. U.S. Patent Publication No. 20040156872 for “Novel nimesulide compositions;” U.S. Patent Publication No. 20040141925 for “Novel triamcinolone compositions;” U.S. Patent Publication No. 20040115134 for “Novel nifedipine compositions;” U.S. Patent Publication No. 20040105889 for “Low viscosity liquid dosage forms;” U.S. Patent Publication No. 20040105778 for “Gamma irradiation of solid nanoparticulate active agents;” U.S. Patent Publication No. 20040101566 for “Novel benzoyl peroxide compositions;” U.S. Patent Publication No. 20040057905 for “Nanoparticulate beclomethasone dipropionate compositions;” U.S. Patent Publication No. 20040033267 for “Nanoparticulate compositions of angiogenesis inhibitors;” U.S. Patent Publication No. 20040033202 for “Nanoparticulate sterol formulations and novel sterol combinations;” U.S. Patent Publication No. 20040018242 for “Nanoparticulate nystatin formulations;” U.S. Patent Publication No. 20040015134 for “Drug delivery systems and methods;” U.S. Patent Publication No. 20030232796 for “Nanoparticulate polycosanol formulations & novel polycosanol combinations;” U.S. Patent Publication No. 20030215502 for “Fast dissolving dosage forms having reduced friability;” U.S. Patent Publication No. 20030185869 for “Nanoparticulate compositions having lysozyme as a surface stabilizer;” U.S. Patent Publication No. 20030181411 for “Nanoparticulate compositions of mitogen-activated protein (MAP) kinase inhibitors;” U.S. Patent Publication No. 20030137067 for “Compositions having a combination of immediate release and controlled release characteristics;” U.S. Patent Publication No. 20030108616 for “Nanoparticulate compositions comprising copolymers of vinyl pyrrolidone and vinyl acetate as surface stabilizers;” U.S. Patent Publication No. 20030095928 for “Nanoparticulate insulin;” U.S. Patent Publication No. 20030087308 for “Method for high through put screening using a small scale mill or microfluidics;” U.S. Patent Publication No. 20030023203 for “Drug delivery systems & methods;” U.S. Patent Publication No. 20020179758 for “System and method for milling materials; and U.S. Patent Publication No. 20010053664 for “Apparatus for sanitary wet milling,” describe nanoparticulate active agent compositions and are specifically incorporated by reference. None of these references describe compositions of nanoparticulate clopidogrel.

Amorphous small particle compositions are described, for example, in U.S. Patent No. 4,783,484 for “Particulate Composition and Use Thereof as Antimicrobial Agent;” U.S. Pat. No. 4,826,689 for “Method for Making Uniformly Sized Particles from Water-Insoluble Organic Compounds;” U.S. Pat. No. 4,997,454 for “Method for Making Uniformly-Sized Particles From Insoluble Compounds;” U.S. Pat. No. 5,741,522 for “Ultrasmall, Non-aggregated Porous Particles of Uniform Size for Entrapping Gas Bubbles Within and Methods;” and U.S. Pat. No. 5,776,496, for “Ultrasmall Porous Particles for Enhancing Ultrasound Back Scatter.”

### SUMMARY

The present invention relates to nanoparticulate compositions comprising clopidogrel, or a salt or derivative thereof. The compositions may include nanoparticulate clopidogrel particles, and may also include at least one surface stabilizer associated with the surface of the clopidogrel. In some embodiments, the surface stabilizer is adsorbed on the surface of the clopidogrel particles.

In some embodiments, the nanoparticulate clopidogrel particles may have an effective average particle size of less than about 2,000 nm. In other embodiments, the effective average particle size of the nanoparticulate clopidogrel particle may be less than about 1900 nm; less than about 1800 nm; less than about 1700 nm; less than about 1600 nm; less than about 1500 nm; less than about 1400 nm; less than about 1300 nm; less than about 1200 nm; less than about 1100 nm; less than about 1000 nm, less than about 900 nm; less than about 800 nm; less than about 700 nm; less than about 600 nm; less than about 500 nm; less than about 400 nm; less than about 300 nm; less than about 250 nm; less than about 200 nm; less than about 100 nm; less than about 75 nm; and in some embodiments, the effective average particle size may be less than about 50 nm.

The nanoparticulate clopidogrel compositions may include clopidogrel particles in a crystalline phase, an amorphous phase, a semi-crystalline phase, a semi-amorphous phase, and mixtures thereof.

Additionally, the nanoparticulate clopidogrel particles may comprise more than one surface stabilizer. For example, the particles may comprise at least one primary and at least one secondary surface stabilizer. The one or more surface stabilizers may include, for example, anionic surface stabilizers, cationic surface stabilizers, non-ionic surface stabilizers, zwitterionic stabilizers or ionic surface stabilizers, or mixtures of these surface stabilizers.

Clopidogrel and at least one surface stabilizer may be present in the pharmaceutical compositions at any suitable ratio (w/w). For example, in some embodiments the pharmaceutical compositions include clopidogrel and the surface stabilizer at a ratio of about 20:1, 15:1, 10:1, 8:1, 7:1, 6:1, 5:1, 4:1, 3:1, 2:1 (w/w), or any range defined by said ratios (for example, but not limited to about 20:1-2:1, about 10:1-4: 1, and about 8:1-5:1). In other embodiments, the surface stabilizer may include from about 0.5% to about 99.999% by weight of the total combined dry weight of clopidogrel and the at least one surface stabilizer, not including other excipients. In other embodiments, the surface stabilizer may include from about 5.0% to about 99.9% by weight; in still other embodiments, the surface stabilizer may include from about 10% to about 99.5% by weight, based on the total combined dry weight of clopidogrel and the at least one surface stabilizer, not including other excipients. Clopidogrel may be present, for example, from about 99.5% to about 0.0001%, from about 95% to about 0.1%, or from about 90% to about 0.5% by weight based on the total combined weight of clopidogrel and the at least one surface stabilizer, not including other excipients. The present compositions contemplate any combination of these exemplary amounts of surface stabilizer and clopidogrel.

The nanoparticulate clopidogrel compositions may be formulated for a variety of administrations. For example, some compositions may be formulated to allow for oral, pulmonary, rectal, colonic, parenteral, intracisternal, intravaginal, intraperitoneal, ocular, otic, local, buccal, nasal or topical administration. Dosage forms of the nanoparticulate clopidogrel compositions may also vary, and may include, for example, liquid dispersions, gels, aerosols, ointments, creams, lyophilized formulation tablets, capsules, controlled release formulations, fast melt formulations, delayed release formulations, extended release formulations, pulsatile release formulations, mixed immediate release formulations, controlled release formulations, bioadhesive formulations or any combination of these dosage forms. In some embodiments, a preferred dosage form may be a solid dosage form, although any pharmaceutically acceptable dosage form may be utilized. In other embodiments, a controlled release formulation may be optimal. In some controlled release formulations, the nanoparticulate clopidogrel particles may be coated with one or more polymeric coatings or may be incorporated in a polymeric material matrix. In other preferred embodiments, the nanoparticulate clopidogrel particles may also be formulated as an injectable, (e.g., intravenous, intramuscular, subcutaneously as a depot) solution for administration immediately prior to or during a cardiac event for the immediate onset of drug therapeutic action as well as improved ease of administration.

Some embodiments may additionally include one or more pharmaceutically acceptable excipients, carriers or a combination of excipients and carriers. Other embodiments may additionally include one or more active agents useful for the treatment of pathologies induced by platelet aggregation. By way of example, but not by way of limitation, exemplary pathologies include thrombotic events, cardiovascular or cerebrovascular diseases, heart attack, stroke, arterial disease; exemplary agents useful for the treatment of pathologies induced by platelet aggregation may include mitotic inhibitors, alkylating agents, anti-metabolites, intercalating antibiotics, growth factor inhibitors, cell cycle inhibitors, enzymes, topoisomerase inhibitors, biological response modifiers, anti-hormones, and anti-androgens.

The present invention also relates to nanoparticulate clopidogrel compositions that may exhibit absorption levels that do not differ significantly when administered under fed as compared to fasting conditions; in some embodiments, administration of the compositions in the fed state may be bioequivalent to the administration of the composition in the fasted state. In some embodiments, the nanoparticulate clopidogrel compositions may produce therapeutic results at a dosage which is less than that of a non-nanoparticulate dosage form of the same clopidogrel. In other embodiments, the nanoparticulate clopidogrel compositions may exhibit one or more of: a greater Cₘₐₓ, a greater AUC, or a lower Tₘₐₓ, when assayed in the plasma of a subject (e.g., a mammal), as compared to a non-nanoparticulate formulation of the same clopidogrel administered at the same dosage.

The present invention also relates to methods of preparing a nanoparticulate clopidogrel or a derivative or salt thereof including clopidogrel particles and at least one surface stabilizer. In some methods, the nanoparticulate compositions may be prepared by contacting clopidogrel particles with at least one surface stabilizer for a time and under conditions sufficient to provide a nanoparticulate clopidogrel composition with an effective average particle size of less than about 2000 nm. In some methods, contacting may include grinding, wet grinding, homogenization, freezing, template emulsion, precipitation, or a combination thereof.

The present invention also relates to methods of treatment of pathologies induced by platelet aggregation such as, for example, cardiovascular or cerebrovascular diseases or conditions; the pathology may be myocardial infarction, blood clot, arterial disease or stroke. In some methods, treatment may involve administering nanoparticulate clopidogrel compositions to a subject, where the composition may include at least one clopidogrel or a derivative or a salt thereof and at least one surface stabilizer, where the particle may have an effective size of less than about 2000 nm. In some methods, the treatment may be prophylactic.

In some methods, the subject may be a survivor of a disease or condition induced by platelet aggregation or may be at increased risk for a disease or condition induced by platelet aggregation. For example, the subject may be a survivor of a thrombotic event or may be at high risk for a thrombotic event; the subject may be a survivor of a myocardial infarction, a blood clot, arterial disease, or a stroke. By way of example but not by way of limitation, the subject may have or may exhibit one or more of the following risk factors: hypertension, smoking, diabetes, high blood cholesterol, overweight, poor diet, arterial disease, age, heredity, gender.

Other methods of treatment using the nanoparticulate compositions of the invention are known to those of skill in the art.

Both the foregoing general description and the following detailed description are exemplary and explanatory and are intended to provide further explanation of the invention as claimed. Other objects, advantages, and novel features will be readily apparent to those skilled in the art from the following detailed description of the invention.

### DETAILED DESCRIPTION

A. Nanoparticulate Clopidogrel Compositions

The present invention is directed to nanoparticulate compositions comprising a clopidogrel, or a salt or derivative thereof. The compositions comprise a clopidogrel, or a salt or derivative thereof, and preferably at least one surface stabilizer adsorbed on or associated with the surface of the drug. The clopidogrel, or salt or derivative thereof, particles have an effective average particle size of less than about 2000 nm.

Advantages of the nanoparticulate clopidogrel formulation of the invention include, but are not limited to: (1) smaller tablet or other solid dosage form size; (2) smaller doses of drug required to obtain the same pharmacological effect as compared to conventional microcrystalline forms of clopidogrel; (3) increased bioavailability as compared to conventional microcrystalline forms of clopidogrel; (4) similar pharmacokinetic profiles of the nanoparticulate clopidogrel in the fed versus fasted state; (5) bioequivalency of the nanoparticulate colpidogrel compositions when administered in the fed versus fasted state; (6) an increased rate of dissolution for the clopidogrel compositions as compared to conventional microcrystalline forms of the same clopidogrel; and (7) the clopidogrel compositions can be used in conjunction with other active agents useful in the prevention and treatment of diseases or conditions caused by, exacerbated by, or involving platelet aggregation.

The present invention also includes nanoparticulate clopidogrel, or a salt or derivative thereof, compositions together with one or more non-toxic physiologically acceptable carriers, adjuvants, or vehicles, collectively referred to as carriers. The compositions can be formulated for parental injection (e.g., intravenous, intramuscular, or subcutaneous), oral administration in solid, liquid, or aerosol form, vaginal, nasal, rectal, ocular, local (powders, ointments, or drops), buccal, intracisternal, intraperitoneal, or topical administrations, and the like.

A preferred dosage form of the invention is a solid dosage form, although any pharmaceutically acceptable dosage form can be utilized. Exemplary solid dosage forms include, but are not limited to, tablets, capsules, sachets, lozenges, powders, pills, or granules, and the solid dosage form can be, for example, a fast melt dosage form, controlled release dosage form, lyophilized dosage form, delayed release dosage form, extended release dosage form, pulsatile release dosage form, mixed immediate release and controlled release dosage form, or a combination thereof. A solid dose tablet formulation is preferred.

The present invention is described herein using several definitions, as set forth below and throughout the application.

The term “effective average particle size of less than about 2000 nm,” as used herein, means that at least about 50% of the nanoparticulate clopidogrel particles have a size of less than about 2000 nm when measured by, for example, sedimentation flow fractionation, photon correlation spectroscopy, light scattering, disk centrifugation, and other techniques known to those of skill in the art.

As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

As used herein, the terms “composition” and “formulation” are used interchangeably.

As used herein, the term “including” has the same meaning as “comprising.”

As used herein with reference to stable nanoparticulate clopidogrel particles “stable” connotes, but is not limited to one or more of the following parameters: (1) the particles do not appreciably flocculate or agglomerate due to interparticle attractive forces or otherwise significantly increase in particle size over time; (2) that the physical structure of the particles is not altered over time, such as by conversion from an amorphous phase to a crystalline phase; (3) that the particles are chemically stable; and/or (4) where the clopiodogrel derivative has not been subject to a heating step at or above the melting point of clopidogrel in the preparation of the nanoparticles of the present invention.

The term “conventional” or “non-nanoparticulate active agent” shall mean an active agent which is solubilized or which has an effective average particle size of greater than about 2000 nm. Nanoparticulate active agents as defined herein have an effective average particle size of less than about 2000 nm.

The phrase “poorly water soluble drugs” as used herein refers to those drugs that have a solubility in water of less than about 30 mg/ml, less than about 20 mg/ml, less than about 10 mg/ml, or less than about 1 mg/ml.

As used herein, the phrase “therapeutically effective amount” shall mean that drug dosage that provides the specific pharmacological response for which the drug is administered in a significant number of subjects in need of such treatment. It is emphasized that a therapeutically effective amount of a drug that is administered to a particular subject in a particular instance will not always be effective in treating the conditions/diseases described herein, even though such dosage is deemed to be a therapeutically effective amount by those of skill in the art. Therapeutically effective amount” as used herein with respect to a clopidogrel dosage shall mean that dosage that provides the specific pharmacological response for which a clopidogrel is administered in a significant number of subjects in need of such treatment. It is to be further understood that clopidogrel dosages are, in particular instances, measured as oral dosages, or with reference to drug levels as measured in blood.

The term “nanoparticulate clopidogrel composition” is understood to include a nanoparticulate clopidogrel composition or formulation, a nanoparticulate clopidogrel salt composition or formulation or a nanoparticulate cloplidogrel derivative composition or formulation. Where one of these terms is used, the other terms are also contemplated; the terms may be used interchangeably.

The term “particulate” as used herein refers to a state of matter which is characterized by the presence of discreet particles, pellets, beads or granules irrespective of their size, shape or morphology. The term “multiparticulate” as used herein means a plurality of discrete, or aggregated, particles, pellets, beads, granules or mixture thereof irrespective of their size, shape or morphology.

As used herein, the term “subject” is used to mean an animal, preferably a mammal, including a human or non-human. The terms patient and subject may be used interchangeably.

B. Preferred Characteristics of the Nanoparticulate Clopidogrel Compositions of the Invention

1. Increased Bioavailability

The nanoparticulate clopidogrel, or a salt or derivative thereof, formulations of the invention are proposed to exhibit increased bioavailability, and require smaller doses as compared to prior conventional clopidogrel formulations. In some embodiments, the nanoparticulate clopidogrel compositions, upon administration to a mammal, produces therapeutic results at a dosage which is less than that of a non-nanoparticulate dosage form of the same clopidogrel. In one embodiment of the invention, the nanoparticulate clopidogrel composition, in accordance with standard pharmacokinetic practice, has a bioavailability that is about 50% greater than a conventional dosage form, about 40% greater, about 30% greater, about 20% greater, or about 10% greater.

2. Improved Pharmacokinetic Profiles

The nanoparticulate clopidogrel, or a salt or derivative thereof, formulations of the invention are proposed to exhibit improved pharmacokinetic profiles in which the maximum plasma concentration of clopidogrel are higher for a given dose than those occurring following administration of a conventional dosage form. In addition, the time to reach maximum plasma concentration will be shorter with nanoparticulate clopidogrel. These changes will improve the therapeutic efficacy of clopidogrel.

The invention preferably provides compositions comprising at least one nanoparticulate clopidogrel or derivative or a salt thereof, having a desirable pharmacokinetic profile when administered to mammalian subjects. The desirable pharmacokinetic profile of the compositions comprising at least one clopidogrel or derivative or a salt thereof and at least one surface stabilizer preferably includes, but is not limited to: (1) a Cₘₐₓ for the clopidogrel or derivative or a salt thereof, when assayed in the plasma of a mammalian subject following administration, that is preferably greater than the Cₘₐₓ for a non-nanoparticulate formulation of the same clopidogrel administered at the same dosage; and/or (2) an AUC for the clopidogrel or derivative or a salt thereof, when assayed in the plasma of a mammalian subject following administration, that is preferably greater than the AUC for a non-nanoparticulate formulation of the same clopidogrel administered at the same dosage; and/or (3) a Tₘₐₓ for the clopidogrel or derivative or a salt thereof, when assayed in the plasma of a mammalian subject following administration, that is preferably less than the Tₘₐₓ for a non-nanoparticulate formulation of the same clopidogrel administered at the same dosage.

For example, in one embodiment, a composition comprising a nanoparticulate clopidogrel or a derivative or salt thereof, and at least one surface stabilizer exhibits in comparative pharmacokinetic testing with a non-nanoparticulate formulation of the same clopidogrel, administered at the same dosage, a Tₘₐₓ not greater than about 90%, not greater than about 80%, not greater than about 70%, not greater than about 60%, not greater than about 50%, not greater than about 30%, not greater than about 25%, not greater than about 20%, not greater than about 15%, not greater than about 10%, or not greater than about 5% of the Tₘₐₓ exhibited by the non-nanoparticulate clopidogrel formulation.

In another embodiment, a composition comprising a nanoparticulate clopidogrel or a derivative or salt thereof, and at least one surface stabilizer exhibits in comparative pharmacokinetic testing with a non-nanoparticulate formulation of the same clopidogrel, administered at the same dosage, a Cₘₐₓ which is at least about 50%, at least about 100%, at least about 200%, at least about 300%, at least about 400%, at least about 500%, at least about 600%, at least about 700%, at least about 800%, at least about 900%, at least about 1000%, at least about 1100%, at least about 1200%, at least about 1300%, at least about 1400%, at least about 1500%, at least about 1600%, at least about 1700%, at least about 1800%, or at least about 1900% greater than the Cₘₐₓ exhibited by the non-nanoparticulate clopidogrel formulation.

In another embodiment, a composition comprising a nanoparticulate clopidogrel or a derivative or salt thereof, and at least one surface stabilizer exhibits in comparative pharmacokinetic testing with a non-nanoparticulate formulation of the same clopidogrel administered at the same dosage, an AUC which is at least about 25%, at least about 50%, at least about 75%, at least about 100%, at least about 125%, at least about 150%, at least about 175%, at least about 200%, at least about 225%, at least about 250%, at least about 275%, at least about 300%, at least about 350%, at least about 400%, at least about 450%, at least about 500%, at least about 550%, at least about 600%, at least about 750%, at least about 700%, at least about 750%, at least about 800%, at least about 850%, at least about 900%, at least about 950%, at least about 1000%, at least about 1050%, at least about 1100%, at least about 1150%, or at least about 1200% greater than the AUC exhibited by the non-nanoparticulate clopidogrel formulation.

The desirable pharmacokinetic profile, as used herein, is the pharmacokinetic profile measured after the initial dose of the clopidogrel or derivative or a salt thereof.

3. The Pharmacokinetic Profiles of the Clopidogrel Compositions of the Invention are not Affected by the Fed or Fasted State of the Subject Ingesting the Compositions

The invention encompasses clopidogrel or derivative or a salt thereof, compositions wherein the pharmacokinetic profile of clopidogrel is not substantially affected by the fed or fasted state of a subject ingesting the composition. This means that there is no substantial difference in the quantity of drug absorbed or the rate of drug absorption when the nanoparticulate clopidogrel compositions are administered in the fed versus the fasted state.

Benefits of a dosage form which substantially eliminates the effect of food include an increase in subject convenience, thereby increasing subject compliance, as the subject does not need to ensure that they are taking a dose either with or without food. This is significant, as with poor subject compliance an increase in the medical condition for which the drug is being prescribed may be observed.

4. Bioequivalency of Clopidogrel Compositions of the Invention When Administered in the Fed Versus the Fasted State

The invention also provides a nanoparticulate clopidogrel or a derivative or a salt thereof, composition in which administration of the composition to a subject in a fasted state is bioequivalent to administration of the composition to a subject in a fed state.

The difference in absorption of the clopidogrel compositions of the invention, when administered in the fed versus the fasted state, preferably is less than about 40%, less than about 35%, less than about 30%, less than about 25%, less than about 20%, less than about 15%, less than about 10%, less than about 5%, or less than about 3%.

In one embodiment of the invention, the invention encompasses compositions comprising at least one nanoparticulate clopidogrel, wherein administration of the composition to a subject in a fasted state is bioequivalent to administration of the composition to a subject in a fed state, in particular as defined by Cmax and AUC guidelines given by the U.S. Food and Drug Administration and the corresponding European regulatory agency (EMEA). Under U.S. FDA guidelines, two products or methods are bioequivalent if the 90% Confidence Intervals (CI) for AUC and Cmax are between 0.80 to 1.25 (Tmax measurements are not relevant to bioequivalence for regulatory purposes). To show bioequivalency between two compounds or administration conditions pursuant to Europe's EMEA guidelines, the 90% CI for AUC must be between 0.80 to 1.25 and the 90% CI for Cmax must between 0.70 to 1.43.

5. Dissolution Profiles of the Clopidogrel Compositions of the Invention

The nanoparticulate clopidogrel, or a salt or derivative thereof, compositions of the invention are proposed to have unexpectedly dramatic dissolution profiles. Rapid dissolution of an administered active agent is preferable, as faster dissolution generally leads to faster onset of action and greater bioavailability. To improve the dissolution profile and bioavailability of the clopidogrel it would be useful to increase the drug's dissolution so that it could attain a level close to 100%.

The clopidogrel compositions of the invention preferably have a dissolution profile in which within about 5 minutes at least about 20% of the composition is dissolved. In other embodiments, at least about 30% or at least about 40% of the clopidogrel composition is dissolved within about 5 minutes. In yet other embodiments, preferably at least about 40%, at least about 50%, at least about 60%, at least about 70%, or at least about 80% of the clopidogrel composition is dissolved within about 10 minutes. In another embodiment, preferably at least about 70%, at least about 80%, at least about 90%, or at least about 100% of the clopidogrel composition is dissolved within 20 minutes.

Dissolution is preferably measured in a medium which is discriminating. Such a dissolution medium will produce two very different dissolution curves for two products having very different dissolution profiles in gastric juices; i.e., the dissolution medium is predictive of in vivo dissolution of a composition. An exemplary dissolution medium is an aqueous medium containing the surfactant sodium lauryl sulfate at 0.025 M. Determination of the amount dissolved can be carried out by spectrophotometry. The rotating blade method (European Pharmacopoeia) can be used to measure dissolution.

6. Redispersability of the Clopidogrel Compositions of the Invention

An additional feature of the clopidogrel, or a salt or derivative thereof, compositions of the invention is that the compositions redisperse such that the effective average particle size of the redispersed clopidogrel particles is less than about 2 microns. This is significant, as if upon administration the clopidogrel compositions of the invention did not redisperse to a substantially nanoparticulate size, then the dosage form may lose the benefits afforded by formulating the clopidogrel into a nanoparticulate size.

This is because nanoparticulate active agent compositions benefit from the small particle size of the active agent; if the active agent does not disperse into the small particle sizes upon administration, them “clumps” or agglomerated active agent particles are formed, owing to the extremely high surface free energy of the nanoparticulate system and the thermodynamic driving force to achieve an overall reduction in free energy. With the formulation of such agglomerated particles, the bioavailability of the dosage form my fall well below that observed with the liquid dispersion form of the nanoparticulate active agent.

Moreover, the nanoparticulate clopidogrel compositions exhibit dramatic redispersion of the nanoparticulate clopidogrel particles upon administration to a mammal, such as a human or animal, as demonstrated by reconstitution/redispersion in a biorelevant aqueous media such that the effective average particle size of the redispersed clopidogrel particles is less than about 2 microns. Such biorelevant aqueous media can be any aqueous media that exhibit the desired ionic strength and pH, which form the basis for the biorelevance of the media. The desired pH and ionic strength are those that are representative of physiological conditions found in the human body. Such biorelevant aqueous media can be, for example, aqueous electrolyte solutions or aqueous solutions of any salt, acid, or base, or a combination thereof, which exhibit the desired pH and ionic strength.

Biorelevant pH is well known in the art. For example, in the stomach, the pH ranges from slightly less than 2 (but typically greater than 1) up to 4 or 5. In the small intestine the pH can range from 4 to 6, and in the colon it can range from 6 to 8. Biorelevant ionic strength is also well known in the art. Fasted state gastric fluid has an ionic strength of about 0.1M while fasted state intestinal fluid has an ionic strength of about 0.14. See e.g., Lindahl et al., “Characterization of Fluids from the Stomach and Proximal Jejunum in Men and Women,” Pharm. Res., 14 (4): 497-502 (1997).

It is believed that the pH and ionic strength of the test solution is more critical than the specific chemical content. Accordingly, appropriate pH and ionic strength values can be obtained through numerous combinations of strong acids, strong bases, salts, single or multiple conjugate acid-base pairs (i.e., weak acids and corresponding salts of that acid), monoprotic and polyprotic electrolytes, etc.

Representative electrolyte solutions can be, but are not limited to, HCl solutions, ranging in concentration from about 0.001 to about 0.1 N, and NaCl solutions, ranging in concentration from about 0.001 to about 0.1 M, and mixtures thereof. For example, electrolyte solutions can be, but are not limited to, about 0.1 N HCl or less, about 0.01 N HCl or less, about 0.001 N HCl or less, about 0.1 M NaCl or less, about 0.01 M NaCl or less, about 0.001 M NaCl or less, and mixtures thereof. Of these electrolyte solutions, 0.01 M HCl and/or 0.1 M NaCl, are most representative of fasted human physiological conditions, owing to the pH and ionic strength conditions of the proximal gastrointestinal tract.

Electrolyte concentrations of 0.001 N HCl, 0.01 N HCl, and 0.1 N HCl correspond to pH 3, pH 2, and pH 1, respectively. Thus, a 0.01 N HCl solution simulates typical acidic conditions found in the stomach. A solution of 0.1 M NaCl provides a reasonable approximation of the ionic strength conditions found throughout the body, including the gastrointestinal fluids, although concentrations higher than 0.1 M may be employed to simulate fed conditions within the human GI tract.

Exemplary solutions of salts, acids, bases or combinations thereof, which exhibit the desired pH and ionic strength, include but are not limited to phosphoric acid/phosphate salts+sodium, potassium and calcium salts of chloride, acetic acid/acetate salts+sodium, potassium and calcium salts of chloride, carbonic acid/bicarbonate salts+sodium, potassium and calcium salts of chloride, and citric acid/citrate salts+sodium, potassium and calcium salts of chloride.

In other embodiments of the invention, the redispersed clopidogrel, or a salt or derivative thereof, particles of the invention (redispersed in water, a biorelevant media, or any other suitable liquid media) have an effective average particle size of less than about less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods. Such methods suitable for measuring effective average particle size are known to a person of ordinary skill in the art.

Redispersibility can be tested using any suitable means known in the art. See e.g., the example sections of U.S. Pat. No. 6,375,986 for “Solid Dose Nanoparticulate Compositions Comprising a Synergistic Combination of a Polymeric Surface Stabilizer and Dioctyl Sodium Sulfosuccinate.”

7. Nanoparticulate Clopidogrel Compositions Used in Conjunction with Other Active Agents

The clopidogrel, or a salt or derivative thereof, compositions of the invention can additionally comprise one or more compounds useful in the prevention and treatment of pathologies induced by platelet aggregation, or the clopidogrel compositions can be administered in conjunction with such a compound. Examples of such compounds include, but are not limited to calcium-entry blocking agents, antianginal agents, cardiac glycosides, vasodilators, antihypertensive agents, blood lipid-lowering agents, antidysrhythmic agents, and antithrombotic agents.

C. Nanoparticulate Clopidogrel Compositions

The invention provides compositions comprising clopidogrel, or a salt or derivative thereof, particles and at least one surface stabilizer. The surface stabilizers preferably are adsorbed on, or associated with, the surface of the clopidogrel particles. Surface stabilizers especially useful herein preferably physically adhere on, or associate with, the surface of the nanoparticulate clopidogrel particles, but do not chemically react with the clopidogrel particles or itself. Individually adsorbed molecules of the surface stabilizer are essentially free of intermolecular cross-linkages.

The present invention also includes clopidogrel, or a salt or derivative thereof, compositions together with one or more non-toxic physiologically acceptable carriers, adjuvants, or vehicles, collectively referred to as carriers. The compositions can be formulated into any pharmaceutically acceptable dosage form, including but not limited to oral and injectable dosage forms. For example, injectable forms may be formulated for parenteral injection (e.g., intravenous, intramuscular, or subcutaneous), oral administration may be formulated in solid, liquid, or aerosol form. Additionally, formulations for vaginal, nasal, rectal, ocular, local (powders, ointments or drops), buccal, intracisternal, intraperitoneal, or topical administration, and the like and also contemplated.

1. Clopidogrel Particles

The clopidogrel particles can comprise clopidogrel or a salt or derivative thereof, such as clopidogrel bisulfate. The clopidogrel particles can be in a crystalline phase, semi-crystalline phase, amorphous phase, semi-amorphous phase, or a combination thereof.

2. Surface Stabilizers

Combinations of more than one surface stabilizers can be used in the invention. Useful surface stabilizers which can be employed in the invention include, but are not limited to, known organic and inorganic pharmaceutical excipients. Such excipients include various polymers, low molecular weight oligomers, natural products, and surfactants. Exemplary surface stabilizers include nonionic, ionic, anionic, cationic, and zwitterionic surfactants or compounds.

Representative examples of surface stabilizers include hydroxypropyl methylcellulose (now known as hypromellose), hydroxypropylcellulose, polyvinylpyrrolidone, sodium lauryl sulfate, dioctylsulfosuccinate, gelatin, casein, lecithin (phosphatides), dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., the commercially available Tweens® such as e.g., Tween 20® and Tween 80® (ICI Speciality Chemicals)); polyethylene glycols (e.g., Carbowaxs 3550® and 934® (Union Carbide)), polyoxyethylene stearates, colloidal silicon dioxide, phosphates, carboxymethylcellulose calcium, carboxymethylcellulose sodium, methylcellulose, hydroxyethylcellulose, hypromellose phthalate, noncrystalline cellulose, magnesium aluminium silicate, triethanolamine, polyvinyl alcohol (PVA), 4-(1,1,3,3-tetramethylbutyl)-phenol polymer with ethylene oxide and formaldehyde (also known as tyloxapol, superione, and triton), poloxamers (e.g., Pluronics F68® and F108®, which are block copolymers of ethylene oxide and propylene oxide); poloxamines (e.g., Tetronic 908®, also known as Poloxamine 908®, which is a tetrafunctional block copolymer derived from sequential addition of propylene oxide and ethylene oxide to ethylenediamine (BASF Wyandotte Corporation, Parsippany, N.J.)); Tetronic 1508® (T-1508) (BASF Wyandotte Corporation), Tritons X-2000®, which is an alkyl aryl polyether sulfonate (Rohm and Haas); Crodestas F-110®, which is a mixture of sucrose stearate and sucrose distearate (Croda Inc.); p-isononylphenoxypoly-(glycidol), also known as Olin-lOG® or Surfactant 10-G® (Olin Chemicals, Stamford, Conn.); Crodestas SL-40® (Croda, Inc.); and SA9OHCO, which is C₁₈H₃₇CH₂(CON(CH₃)—CH₂(CHOH)₄(CH₂0H)₂ (Eastman Kodak Co.); decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; PEG-phospholipid, PEG-cholesterol, PEG-cholesterol derivative, PEG-vitamin A, PEG-vitamin E, lysozyme, random copolymers of vinyl pyrrolidone and vinyl acetate such as Plasdone® S630, and the like.

Examples of useful cationic surface stabilizers include, but are not limited to, polymers, biopolymers, polysaccharides, cellulosics, alginates, phospholipids, and nonpolymeric compounds, such as zwitterionic stabilizers, poly-n-methylpyridinium, anthryul pyridinium chloride, cationic phospholipids, chitosan, polylysine, polyvinylimidazole, polybrene, polymethylmethacrylate trimethylammoniumbromide bromide (PMMTMABr), hexyldesyltrimethylammonium bromide (HDMAB), and polyvinylpyrrolidone-2-dimethylaminoethyl methacrylate dimethyl sulfate.

Other useful cationic stabilizers include, but are not limited to, cationic lipids, sulfonium, phosphonium, and quaternary ammonium compounds, such as stearyltrimethylammonium chloride, benzyl-di(2-chloroethyl)ethylammonium bromide, coconut trimethyl ammonium chloride or bromide, coconut methyl dihydroxyethyl ammonium chloride or bromide, decyl triethyl ammonium chloride, decyl dimethyl hydroxyethyl ammonium chloride or bromide, C₁₂₋₁₅dimethyl hydroxyethyl ammonium chloride or bromide, coconut dimethyl hydroxyethyl ammonium chloride or bromide, myristyl trimethyl ammonium methyl sulphate, lauryl dimethyl benzyl ammonium chloride or bromide, lauryl dimethyl (ethenoxy)₄ ammonium chloride or bromide, N-alkyl (C₁₂₋₁₈)dimethylbenzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts and dialkyl-dimethylammonium salts, lauryl trimethyl ammonium chloride, ethoxylated alkyamidoalkyldialkylammonium salt and/or an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride and dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂, C₁₅, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride (ALIQUAT 336™), POLYQUAT 10™, tetrabutylammonium bromide, benzyl trimethylammonium bromide, choline esters (such as choline esters of fatty acids), benzalkonium chloride, stearalkonium chloride compounds (such as stearyltrimonium chloride and Di-stearyldimonium chloride), cetyl pyridinium bromide or chloride, halide salts of quaternized polyoxyethylalkylamines, MIRAPOL™ and ALKAQUAT™ (Alkaril Chemical Company), alkyl pyridinium salts; amines, such as alkylamines, dialkylamines, alkanolamines, polyethylenepolyamines, N,N-dialkylaminoalkyl acrylates, and vinyl pyridine, amine salts, such as lauryl amine acetate, stearyl amine acetate, alkylpyridinium salt, and alkylimidazolium salt, and amine oxides; imide azolinium salts; protonated quaternary acrylamides; methylated quaternary polymers, such as poly[diallyl dimethylammonium chloride] and poly-[N-methyl vinyl pyridinium chloride]; and cationic guar.

Such exemplary cationic surface stabilizers and other useful cationic surface stabilizers are described in J. Cross and E. Singer, Cationic Surfactants: Analytical and Biological Evaluation (Marcel Dekker, 1994); P. and D. Rubingh (Editor), Cationic Surfactants: Physical Chemistry (Marcel Dekker, 1991); and J. Richmond, Cationic Surfactants: Organic Chemistry, (Marcel Dekker, 1990).

Nonpolymeric surface stabilizers are any nonpolymeric compound, such benzalkonium chloride, a carbonium compound, a phosphonium compound, an oxonium compound, a halonium compound, a cationic organometallic compound, a quaternary phosphorous compound, a pyridinium compound, an anilinium compound, an ammonium compound, a hydroxylammonium compound, a primary ammonium compound, a secondary ammonium compound, a tertiary ammonium compound, and quarternary ammonium compounds of the formula NR₁R₂R₃R₄⁽⁺⁾. For compounds of the formula NR₁R₂R₃R₄⁽⁺⁾:
(i) none of R₁-R₄ are CH₃;
(ii) one of R₁-R₄ is CH₃;
(iii) three of R₁-R₄ are CH₃;
(iv) all of R₁-R₄ are CH₃;
(v) two of R₁-R₄ are CH₃, one of R₁-R₄ is C₆H₅CH₂, and one of R₁-R₄ is an alkyl chain of seven carbon atoms or less;
(vi) two of R₁-R₄ are CH₃, one of R₁-R₄ is C₆H₅CH₂, and one of R₁-R₄ is an alkyl chain of nineteen carbon atoms or more;
(vii) two of R₁-R₄ are CH₃ and one of R₁-R₄ is the group C₆H₅(CH₂)ₙ, where n>1;
(viii) two of R₁-R₄ are CH₃, one of R₁-R₄ is C₆H₅CH₂, and one of R₁-R₄ comprises at least one heteroatom;
(ix) two of R₁-R₄ are CH₃, one of R₁-R₄ is C₆H₅CH₂, and one of R₁-R₄ comprises at least one halogen;
(x) two of R₁-R₄ are CH₃, one of R₁-R₄ is C₆H₅CH₂, and one of R₁-R₄ comprises at least one cyclic fragment;
(xi) two of R₁-R₄ are CH₃ and one of R₁-R₄ is a phenyl ring; or
(xii) two of R₁-R₄ are CH₃ and two of R₁-R₄ are purely aliphatic fragments.

Such compounds include, but are not limited to, behenalkonium chloride, benzethonium chloride, cetylpyridinium chloride, behentrimonium chloride, lauralkonium chloride, cetalkonium chloride, cetrimonium bromide, cetrimonium chloride, cethylamine hydrofluoride, chlorallylmethenamine chloride (Quatemium-15), distearyldimonium chloride (Quaternium-5), dodecyl dimethyl ethylbenzyl ammonium chloride(Quaternium-14), Quaternium-22, Quaternium-26, Quaternium-18 hectorite, dimethylaminoethylchloride hydrochloride, cysteine hydrochloride, diethanolammonium POE (10) oletyl ether phosphate, diethanolammonium POE (3)oleyl ether phosphate, tallow alkonium chloride, dimethyl dioctadecylammoniumbentonite, stearalkonium chloride, domiphen bromide, denatonium benzoate, myristalkonium chloride, laurtrimonium chloride, ethylenediamine dihydrochloride, guanidine hydrochloride, pyridoxine HCl, iofetamine hydrochloride, meglumine hydrochloride, methylbenzethonium chloride, myrtrimonium bromide, oleyltrimonium chloride, polyquaternium-1, procainehydrochloride, cocobetaine, stearalkonium bentonite, stearalkoniumhectonite, stearyl trihydroxyethyl propylenediamine dihydrofluoride, tallowtrimonium chloride, and hexadecyltrimethyl ammonium bromide.

The surface stabilizers are commercially available and/or can be prepared by techniques known in the art. Most of these surface stabilizers are known pharmaceutical excipients and are described in detail in the Handbook of Pharmaceutical Excipients, published jointly by the American Pharmaceutical Association and The Pharmaceutical Society of Great Britain (The Pharmaceutical Press, 2000), specifically incorporated by reference.

3. Other Pharmaceutical Excipients

Pharmaceutical compositions according to the invention may also comprise one or more binding agents, filling agents, lubricating agents, suspending agents, sweeteners, flavoring agents, preservatives, buffers, wetting agents, disintegrants, effervescent agents, and other excipients. Such excipients are known in the art.

Examples of filling agents are lactose monohydrate, lactose anhydrous, and various starches; examples of binding agents are various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCC™).

Suitable lubricants, including agents that act on the flowability of the powder to be compressed, are colloidal silicon dioxide, such as Aerosil® 200, talc, stearic acid, magnesium stearate, calcium stearate, and silica gel.

Examples of sweeteners are any natural or artificial sweetener, such as sucrose, xylitol, sodium saccharin, cyclamate, aspartame, and acsulfame. Examples of flavoring agents are Magnasweet® (trademark of MAFCO), bubble gum flavor, and fruit flavors, and the like.

Examples of preservatives are potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quarternary compounds such as benzalkonium chloride.

Suitable diluents include pharmaceutically acceptable inert fillers, such as microcrystalline cellulose, lactose, dibasic calcium phosphate, saccharides, and/or mixtures of any of the foregoing. Examples of diluents include microcrystalline cellulose, such as Avicel®0 5 PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

Examples of effervescent agents are effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

Aqueous suspensions comprising the nanoparticulate clopidogrel can be in admixture with excipients suitable for the manufacture of aqueous suspensions. Such excipients are suspending agents, for example, sodium carboxymethylcellulose, methylcellulose, hydroxy-propylmethylcellulose, sodium alginate, polyvinylpyrrolidone, gum tragacanth and gum acadia.

Examples of buffers are phosphate buffers, citrate buffers and buffers made from other organic acids.

Examples of wetting or dispersing agents are a naturally-occurring phosphatide, for example, lecithin or condensation products of n-alkylene oxide with fatty acids, for example, polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol mono-oleate, or condensation products of ethylene oxide with partial esters derived from fatty acids and hexitol anhydrides, for example, polyethylene sorbitan monooleate.

4. Nanoparticulate Clopidogrel Particle Size

The compositions of the invention contain nanoparticulate clopidogrel, or a salt or derivative thereof, particles which have an effective average particle size of less than about 2000 nm (i.e., 2 microns), less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 rn, less than about 900 run, less than about 800 nm, less than about 700 nm, less than about 600 nm, less than about 500 nm, less than about 400 nm, less than about 300 nm, less than about 250 nm, less than about 200 run, less than about 150 nm, less than about 100 nm, less than about 75 nm, or less than about 50 nm, as measured by light-scattering methods, microscopy, or other appropriate methods.

By “an effective average particle size of less than about 2000 nm” it is meant that at least 50% of the clopidogrel particles have a particle size of less than the effective average, by weight (or by other suitable means, such as volume, number, etc.), i.e., less than about 2000 nm, 1900 nm, 1800 nm, etc., when measured by the above-noted techniques. In other embodiments of the invention, at least about 60%, at least about 70%, at least about 80%, at least about 90%, at least about 95%, or at least about 99% of the clopidogrel particles have a particle size of less than the effective average, i.e., less than about 2000 nm, 1900 nm, 1800 nm, 1700 nm, etc.

In the present invention, the value for D50 of a nanoparticulate clopidogrel composition is the particle size below which 50% of the clopidogrel particles fall, by weight. Similarly, D90 is the particle size below which 90% of the clopidogrel particles fall, by weight.

5. Concentration of Clopidogrel and Surface Stabilizers

The relative amounts of clopidogrel, or a salt or derivative thereof, and one or more surface stabilizers can vary widely. The optimal amount of the individual components can depend, for example, upon the particular clopidogrel selected, the hydrophilic lipophilic balance (HLB), melting point, and the surface tension of water solutions of the stabilizer, etc.

The concentration of the clopidogrel can vary from about 99.5% to about 0.001%, from about 95% to about 0.1%, or from about 90% to about 0.5%, by weight, based on the total combined weight of the clopidogrel and at least one surface stabilizer, not including other excipients.

The concentration of the at least one surface stabilizer can vary from about 0.5% to about 99.999%, from about 5.0% to about 99.9%, or from about 10% to about 99.5%, by weight, based on the total combined dry weight of the clopidogrel and at least one surface stabilizer, not including other excipients.

6. Exemplary Nanoparticulate Clopidogrel Bisulfate Tablet Formulations

Several exemplary clopidogrel bisulfate tablet formulations are given below. These examples are not intended to limit the claims in any respect, but rather to provide exemplary tablet formulations of clopidogrel bisulfate which can be utilized in the methods of the invention. Such exemplary tablets can also comprise a coating agent.

TABLE #1
Exemplary Nanoparticulate
Clopidogrel Bisulfate Tablet Formulation #1

| Component | g/Kg |
|---|---|
| Clopidogrel Bisulfate | about 50 to about 500 |
| Hypromellose, USP | about 10 to about 70 |
| Docusate Sodium, USP | about 1 to about 10 |
| Sucrose, NF | about 100 to about 500 |
| Sodium Lauryl Sulfate, NF | about 1 to about 40 |
| Lactose Monohydrate, NF | about 50 to about 400 |
| Silicified Microcrystalline Cellulose | about 50 to about 300 |
| Crospovidone, NF | about 20 to about 300 |
| Magnesium Stearate, NF | about 0.5 to about 5 |

TABLE #2
Exemplary Nanoparticulate
Clopidogrel Bisulfate Tablet Formulation #2

| Component | g/Kg |
|---|---|
| Clopidogrel Bisulfate | about 100 to about 300 |
| Hypromellose, USP | about 30 to about 50 |
| Docusate Sodium, USP | about 0.5 to about 10 |
| Sucrose, NF | about 100 to about 300 |
| Sodium Lauryl Sulfate, NF | about 1 to about 30 |
| Lactose Monohydrate, NF | about 100 to about 300 |
| Silicified Microcrystalline Cellulose | about 50 to about 200 |
| Crospovidone, NF | about 50 to about 200 |
| Magnesium Stearate, NF | about 0.5 to about 5 |

TABLE #3
Exemplary Nanoparticulate
Clopidogrel Bisulfate Tablet Formulation #3

| Component | g/Kg |
|---|---|
| Clopidogrel Bisulfate | about 200 to about 225 |
| Hypromellose, USP | about 42 to about 46 |
| Docusate Sodium, USP | about 2 to about 6 |
| Sucrose, NF | about 200 to about 225 |
| Sodium Lauryl Sulfate, NF | about 12 to about 18 |
| Lactose Monohydrate, NF | about 200 to about 205 |
| Silicified Microcrystalline Cellulose | about 130 to about 135 |
| Crospovidone, NF | about 112 to about 118 |
| Magnesium Stearate, NF | about 0.5 to about 3 |

TABLE #4
Exemplary Nanoparticulate
Clopidogrel Bisulfate Tablet Formulation #4

| Component | g/Kg |
|---|---|
| Clopidogrel Bisulfate | about 119 to about 224 |
| Hypromellose, USP | about 42 to about 46 |
| Docusate Sodium, USP | about 2 to about 6 |
| Sucrose, NF | about 119 to about 224 |
| Sodium Lauryl Sulfate, NF | about 12 to about 18 |
| Lactose Monohydrate, NF | about 119 to about 224 |
| Silicified Microcrystalline Cellulose | about 129 to about 134 |
| Crospovidone, NF | about 112 to about 118 |
| Magnesium Stearate, NF | about 0.5 to about 3 |

D. Methods of Making Nanoparticulate Clopidogrel Compositions

The nanoparticulate clopidogrel, or a salt or derivative thereof, compositions can be made using any suitable method known in the art such as, for example, milling, homogenization, precipitation, freezing, or template emulsion techniques. Exemplary methods of making nanoparticulate compositions are described in the '684 patent.

Exemplary methods of making nanoparticulate compositions are also described in U.S. Pat. No. 5,518,187 for “Method of Grinding Pharmaceutical Substances;” U.S. Pat. No. 5,718,388 for “Continuous Method of Grinding Pharmaceutical Substances;” U.S. Pat. No. 5,862,999 for “Method of Grinding Pharmaceutical Substances;” U.S. Pat. No. 5,665,331 for “Co-Microprecipitation of Nanoparticulate Pharmaceutical Agents with Crystal Growth Modifiers;” U.S. Pat. No. 5,662,883 for “Co-Microprecipitation of Nanoparticulate Pharmaceutical Agents with Crystal Growth Modifiers;” U.S. Pat. No. 5,560,932 for “Microprecipitation of Nanoparticulate Pharmaceutical Agents;” U.S. Pat. No. 5,543,133 for “Process of Preparing X-Ray Contrast Compositions Containing Nanoparticles;” U.S. Pat. No. 5,534,270 for “Method of Preparing Stable Drug Nanoparticles;” U.S. Pat. No. 5,510,118 for “Process of Preparing Therapeutic Compositions Containing Nanoparticles;” and U.S. Pat. No. 5,470,583 for “Method of Preparing Nanoparticle Compositions Containing Charged Phospholipids to Reduce Aggregation,” all of which are specifically incorporated by reference.

An exemplary method of preparing the nanoparticulate clopidogrel formulations of the invention comprises the steps of: (1) dispersing the desired dosage amount of a clopidogrel in a liquid dispersion media in which the drug is poorly soluble; and (2) mechanically reducing the particle size of the clopidogrel to an effective average particle size of less than about 2000 nm. A surface stabilizer can be added to the dispersion media either before, during, or after particle size reduction of the clopidogrel. Preferably, the dispersion media used for the size reduction process is aqueous, although any dispersion media in which the clopidogrel is poorly soluble can be used, such as safflower oil, ethanol, t-butanol, glycerin, polyethylene glycol (PEG), hexane, or glycol.

Using a particle size reduction method, the particle size of the clopidogrel is reduced to an effective average particle size of less than about 2000 nm. Effective methods of providing mechanical force for particle size reduction of the clopidogrel include methods such as for example, ball milling, media milling, and homogenization, for example, with a Microfluidizer® (Microfluidics Corp.).

The resultant nanoparticulate clopidogrel compositions or dispersions can be utilized in solid or liquid dosage formulations, such as liquid dispersions, gels, aerosols, ointments, creams, controlled release formulations, fast melt formulations, lyophilized formulations, tablets, capsules, delayed release formulations, extended release formulations, pulsatile release formulations, mixed immediate release and controlled release formulations, etc.

1. Milling to Obtain Nanoparticulate Clopidogrel Dispersions

Milling a clopidogrel, or a salt or derivative thereof, to obtain a nanoparticulate dispersion comprises dispersing the clopidogrel particles in a liquid dispersion medium in which the clopidogrel is poorly soluble, followed by applying mechanical means in the presence of grinding media to reduce the particle size of the clopidogrel to the desired effective average particle size. The dispersion medium can be, for example, water, safflower oil, ethanol, t-butanol, glycerin, polyethylene glycol (PEG), hexane, or glycol. A preferred dispersion medium is water.

The clopidogrel particles can be reduced in size in the presence of at least one surface stabilizer. Alternatively, clopidogrel particles can be contacted with one or more surface stabilizers after attrition. Other compounds, such as a diluent, can be added to the clopidogrel/surface stabilizer composition during the size reduction process. Dispersions can be manufactured continuously or in a batch mode.

The clopidogrel particles can be added to a liquid media in which it is essentially insoluble to form a premix. The surface stabilizer can be present in the premix or it can be added to the clopidogrel dispersion following particle size reduction. The premix can be used directly by subjecting it to mechanical means to reduce the average clopidogrel particle size in the dispersion to less than about 2000 nm. It is preferred that the premix be used directly when a ball mill is used for attrition. Alternatively, the clopidogrel and at least one surface stabilizer can be dispersed in the liquid media using suitable agitation, e.g., a Cowles type mixer, until a homogeneous dispersion is observed in which there are no large agglomerates visible to the naked eye. It is preferred that the premix be subjected to such a pre-milling dispersion step when a re-circulating media mill is used for attrition.

The mechanical means applied to reduce the clopidogrel particle size can take the form of a dispersion mill. Suitable dispersion mills include a ball mill, an attritor mill, a vibratory mill, and media mills such as a sand mill and a bead mill. A media mill is preferred due to the relatively shorter milling time required to provide the desired reduction in particle size.

Media milling is a high energy milling process. Clopidogrel, surface stabilizer, and liquid are placed in a reservoir and re-circulated in a chamber comprising grinding media and a rotating shaft/impeller. The rotating shaft agitates the grinding media which subjects the clopidogrel to impaction and sheer forces, thereby reducing the clopidogrel particle size. For media milling, the apparent viscosity of the premix is preferably from about 100 to about 1000 centipoise, and for ball milling the apparent viscosity of the premix is preferably from about 1 up to about 100 centipoise. Such ranges tend to afford an optimal balance between efficient particle size reduction and media erosion.

Ball milling is a low energy milling process that uses milling media, drug, stabilizer, and liquid. The materials are placed in a milling vessel that is rotated at optimal speed such that the media cascades and reduces the drug particle size by impaction. The media used must have a high density as the energy for the particle reduction is provided by gravity and the mass of the attrition media.

The attrition time can vary widely and depends primarily upon the particular mechanical means and processing conditions selected. For ball mills, processing times of up to five days or longer may be required. Alternatively, processing times of less than 1 day (residence times of one minute up to several hours) are possible with the use of a high shear media mill.

The clopidogrel particles can be reduced in size at a temperature which does not significantly degrade the clopidogrel molecule. Processing temperatures of less than about 30 to less than about 40° C. are ordinarily preferred. If desired, the processing equipment can be cooled with conventional cooling equipment. Control of the temperature, e.g., by jacketing or immersion of the milling chamber in ice water, is contemplated. Generally, the method of the invention is conveniently carried out under conditions of ambient temperature and at processing pressures which are safe and effective for the milling process. Ambient processing pressures are typical of ball mills, attritor mills, and vibratory mills.

Grinding Media

The grinding media for the particle size reduction step can be selected from rigid media preferably spherical or particulate in form having an average size less than about 3 mm and, more preferably, less than about 1 mm. Such media desirably can provide the particles of the invention with shorter processing times and impart less wear to the milling equipment. The selection of material for the grinding media is not believed to be critical. Zirconium oxide, such as 95% ZrO stabilized with magnesia, zirconium silicate, ceramic, stainless steel, titania, alumina, 95% ZrO stabilized with yttrium, glass grinding media, and polymeric grinding media are exemplary grinding materials.

The grinding media can comprise particles that are preferably substantially spherical in shape, e.g., beads, consisting essentially of polymeric resin or other suitable material. Alternatively, the grinding media can comprise a core having a coating of a polymeric resin adhered thereon. The polymeric resin can have a density from about 0.8 to about 3.0 g/cm3.

In general, suitable polymeric resins are chemically and physically inert, substantially free of metals, solvent, and monomers, and of sufficient hardness and friability to enable them to avoid being chipped or crushed during grinding. Suitable polymeric resins include crosslinked polystyrenes, such as polystyrene crosslinked with divinylbenzene; styrene copolymers; polycarbonates; polyacetals, such as Delrin® (E. I. du Pont de Nemours and Co.); vinyl chloride polymers and copolymers; polyurethanes; polyamides; poly(tetrafluoroethylenes), e.g., Teflon® (E. I. du Pont de Nemours and Co.), and other fluoropolymers; high density polyethylenes; polypropylenes; cellulose ethers and esters such as cellulose acetate; polyhydroxymethacrylate; polyhydroxyethyl acrylate; and silicone-containing polymers such as polysiloxanes and the like. The polymer can be biodegradable. Exemplary biodegradable polymers include poly(lactides), poly(glycolide) copolymers of lactides and glycolide, polyanhydrides, poly(hydroxyethyl methacylate), poly(imino carbonates), poly(N-acylhydroxyproline)esters, poly(N-palmitoyl hydroxyproline) esters, ethylene-vinyl acetate copolymers, poly(orthoesters), poly(caprolactones), and poly(phosphazenes). For biodegradable polymers, contamination from the media itself advantageously can metabolize in vivo into biologically acceptable products that can be eliminated from the body.

The grinding media preferably ranges in size from about 0.01 to about 3 mm. For fine grinding, the grinding media is preferably from about 0.02 to about 2 mm, and more preferably from about 0.03 to about 1 mm in size.

In a preferred grinding process the clopidogrel particles are made continuously. Such a method comprises continuously introducing the clopidogrel into a milling chamber, contacting the compounds with grinding media while in the chamber to reduce the particle size, and continuously removing the nanoparticulate clopidogrel from the milling chamber.

The grinding media is separated from the milled nanoparticulate clopidogrel using conventional separation techniques, in a secondary process such as by simple filtration, sieving through a mesh filter or screen, and the like. Other separation techniques such as centrifugation may also be employed.

2. Precipitation to Obtain Nanoparticulate Clopidogrel Compositions

Another method of forming the desired nanoparticulate clopidogrel, or a salt or derivative thereof, composition is by microprecipitation. This is a method of preparing stable dispersions of poorly soluble active agents in the presence of one or more surface stabilizers and one or more colloid stability enhancing surface active agents free of any trace toxic solvents or solubilized heavy metal impurities. Such a method comprises, for example: (1) dissolving the clopidogrel in a suitable solvent; (2) adding the formulation from step (1) to a solution comprising at least one surface stabilizer; and (3) precipitating the formulation from step (2) using an appropriate non-solvent. The method can be followed by removal of any formed salt, if present, by dialysis or diafiltration and concentration of the dispersion by conventional means.

3. Homogenization to Obtain Nanoparticulate Clopidogrel Compositions

Homogenization is a technique that does not use milling media. Clopidogrel, surface stabilizer, and liquid (or drug and liquid with the surface stabilizer added after particle size reduction) constitute a process stream propelled into a process zone, which in the Microfluidizer® is called the Interaction Chamber. The product to be treated is inducted into the pump, and then forced out. The priming valve of the Microfluidizer® purges air out of the pump. Once the pump is filled with product, the priming valve is closed and the product is forced through the interaction chamber. The geometry of the interaction chamber produces powerful forces of sheer, impact, and cavitation which are responsible for particle size reduction. Specifically, inside the interaction chamber, the pressurized product is split into two streams and accelerated to extremely high velocities. The formed jets are then directed toward each other and collide in the interaction zone. The resulting product has very fine and uniform particle or droplet size. The Microfluidizer® also provides a heat exchanger to allow cooling of the product.

U.S. Pat. No. 5,510,118, which is specifically incorporated by reference, refers to a process using a Microfluidizer.® Such a method comprises dispersing particles of a clopidogrel, or a salt or derivative thereof, in a liquid dispersion medium, followed by subjecting the dispersion to homogenization to reduce the particle size of a clopidogrel to the desired effective average particle size. The clopidogrel particles may be reduced in size in the presence of at least one surface stabilizer. Alternatively, the clopidogrel particles may be contacted with one or more surface stabilizers either before or after attrition. Other compounds, such as a diluent, can be added to the clopidogrel/surface stabilizer composition either before, during, or after the size reduction process. Dispersions can be manufactured continuously or in a batch mode.

4. Cryogenic Methodologies to Obtain Nanoparticulate Clopidogrel Compositions

Another method of forming the desired nanoparticulate clopidogrel, or a salt or derivative thereof, composition is by spray freezing into liquid (SFL). This technology comprises an organic or organoaqueous solution of clopidogrel with stabilizers, which is injected into a cryogenic liquid, such as liquid nitrogen. The droplets of the clopidogrel solution freeze at a rate sufficient to minimize crystallization and particle growth, thus formulating nanostructured clopidogrel particles. Depending on the choice of solvent system and processing conditions, the nanoparticulate clopidogrel particles can have varying particle morphology. In the isolation step, the nitrogen and solvent are removed under conditions that avoid agglomeration or ripening of the clopidogrel particles.

As a complementary technology to SFL, ultra rapid freezing (URF) may also be used to created equivalent nanostructured clopidogrel particles with greatly enhanced surface area.

URF comprises an organic or organoaqueous solution of clopidogrel with stabilizers onto a cryogenic substrate.

5. Emulsion Methodologies to Obtain Nanoparticulate Clopidogrel Compositions

Another method of forming the desired nanoparticulate clopidogrel, or a salt or derivative thereof, composition is by template emulsion. Template emulsion creates nanostructured clopidogrel particles with controlled particle size distribution and rapid dissolution performance. The method comprises an oil-in-water emulsion that is prepared, then swelled with a non-aqueous solution comprising the clopidogrel and stabilizers. The particle size distribution of the clopidogrel particles is a direct result of the size of the emulsion droplets prior to loading with the clopidogrel a property which can be controlled and optimized in this process. Furthermore, through selected use of solvents and stabilizers, emulsion stability is achieved with no or suppressed Ostwald ripening. Subsequently, the solvent and water are removed, and the stabilized nanostructured clopidogrel particles are recovered. Various clopidogrel particles morphologies can be achieved by appropriate control of processing conditions.

Published International Patent Application No. WO 97/144407 to Pace et al., published Apr. 24, 1997, discloses particles of water insoluble biologically active compounds with an average size of 100 nm to 300 nm that are prepared by dissolving the compound in a solution and then spraying the solution into compressed gas, liquid or supercritical fluid in the presence of appropriate surface modifiers.

E. Methods of Using the Nanoparticulate Clopidogrel Compositions of the Invention

The invention provides a method of increasing bioavailability of a clopidogrel, or a salt or derivative thereof, in a subject. Such a method comprises orally administering to a subject an effective amount of a composition comprising a nanoparticulate clopidogrel.

In addition, the nanoparticulate clopidogrel compositions, in accordance with standard pharmacokinetic practice, preferably produces a maximum blood plasma concentration profile in less than about 6 hours, less than about 5 hours, less than about 4 hours, less than about 3 hours, less than about 2 hours, less than about 1 hour, or less than about 30 minutes after the initial dose of the composition.

The compositions of the invention are useful in the prevention and treatment of pathological states induced by platelet aggregation. Such pathological states include, but are not limited to, cardiovascular and cerebrovascular system diseases such as the thromboembolic disorders associated with atherosclerosis or with diabetes such as unstable angina, cerebral attack, restenosis following angioplasty, endarterectomy or fitting of metallic endovascular prostheses, with rethrombosis following thrombolysis, with infarction, with dementia of ischemic origin, with peripheral arterial diseases, with haemodialyses, with auricular fibrillations or during the use of vascular prostheses or aortocoronary bypasses or in relation to stable or unstable angor. Preferably, the compositions of the invention are useful in the prevention and treatment of cardiovascular disease.

The clopidogrel, or a salt or derivative thereof, compounds of the invention can be administered to a subject via any conventional means including, but not limited to, orally, rectally, ocularly, parenterally (e.g., intravenous, intramuscular, or subcutaneous), intracisternally, pulmonary, intravaginally, intraperitoneally, locally (e.g., powders, ointments or drops), or as a buccal or nasal spray. As used herein, the term “subject” is used to mean an animal, preferably a mammal, including a human or non-human. The terms patient and subject may be used interchangeably.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents, or vehicles including water, ethanol, polyols (propyleneglycol, polyethylene-glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants.

The nanoparticulate clopidogrel, or a salt or derivative thereof, compositions may also contain adjuvants such as preserving, wetting, emulsifying, and dispensing agents. Prevention of the growth of microorganisms can be ensured by various antibacterial and antifungal agents, such as parabens, chlorobutanol, phenol, sorbic acid, and the like. It may also be desirable to include isotonic agents, such as sugars, sodium chloride, and the like. Prolonged absorption of the injectable pharmaceutical form can be brought about by the use of agents delaying absorption, such as aluminum monostearate and gelatin.

Solid dosage forms for oral administration include, but are not limited to, capsules, tablets, pills, powders, and granules. In such solid dosage forms, the active agent is admixed with at least one of the following: (a) one or more inert excipients (or carriers), such as sodium citrate or dicalcium phosphate; (b) fillers or extenders, such as starches, lactose, sucrose, glucose, mannitol, and silicic acid; (c) binders, such as carboxymethylcellulose, alignates, gelatin, polyvinylpyrrolidone, sucrose, and acacia; (d) humectants, such as glycerol; (e) disintegrating agents, such as agar-agar, calcium carbonate, potato or tapioca starch, alginic acid, certain complex silicates, and sodium carbonate; (f) solution retarders, such as paraffin; (g) absorption accelerators, such as quaternary ammonium compounds; (h) wetting agents, such as cetyl alcohol and glycerol monostearate; (i) adsorbents, such as kaolin and bentonite; and (j) lubricants, such as talc, calcium stearate, magnesium stearate, solid polyethylene glycols, sodium lauryl sulfate, or mixtures thereof. For capsules, tablets, and pills, the dosage forms may also comprise buffering agents.

Liquid dosage forms for oral administration include pharmaceutically acceptable emulsions, solutions, suspensions, syrups, and elixirs. In addition to a clopidogrel, the liquid dosage forms may comprise inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, oils, such as cottonseed oil, groundnut oil, corn germ oil, olive oil, castor oil, and sesame oil, glycerol, tetrahydrofurfuryl alcohol, polyethyleneglycols, fatty acid esters of sorbitan, or mixtures of these substances, and the like.

Besides such inert diluents, the composition can also include adjuvants, such as wetting agents, emulsifying and suspending agents, sweetening, flavoring, and perfuming agents.

One of ordinary skill will appreciate that effective amounts of a clopidogrel can be determined empirically and can be employed in pure form or, where such forms exist, in pharmaceutically acceptable salt, ester, or prodrug form. Actual dosage levels of a clopidogrel in the nanoparticulate compositions of the invention may be varied to obtain an amount of a clopidogrel that is effective to obtain a desired therapeutic response for a particular composition and method of administration. The selected dosage level therefore depends upon the desired therapeutic effect, the route of administration, the potency of the administered clopidogrel, the desired duration of treatment, and other factors.

Dosage unit compositions may contain such amounts of such submultiples thereof as may be used to make up the daily dose. It will be understood, however, that the specific dose level for any particular patient will depend upon a variety of factors: the type and degree of the cellular or physiological response to be achieved; activity of the specific agent or composition employed; the specific agents or composition employed; the age, body weight, general health, sex, and diet of the patient; the time of administration, route of administration, and rate of excretion of the agent; the duration of the treatment; drugs used in combination or coincidental with the specific agent; and like factors well known in the medical arts.

The following example is for illustrative purposes only, and should not be interpreted as restricting the spirit and scope of the invention, as defined by the scope of the claims that follow. All references cited herein, including U.S. patents, are specifically incorporated by reference.

### EXAMPLE 1

The purpose of this example was to describe how a nanoparticulate clopidogrel composition could be prepared.

An aqueous dispersion of clopidogrel bisulfate can be combined with one or more surface stabilizers, followed by milling in a 10 ml chamber of a NanoMill® 0.01 (NanoMill Systems, King of Prussia, Pa.; see e.g., U.S. Pat. No. 6,431,478), along with 500 micron PolyMill® attrition media (Dow Chemical) (89% media load). The composition can be milled for a suitable period of time, such as about 60 min. at a speed of 2500.

The milled composition can be harvested and analyzed via microscopy. Microscopy can be done, for example, using a Lecia DM5000B and Lecia CTR 5000 light source (Laboratory Instruments and Supplies Ltd., Ashbourne Co., Meath, Ireland). Microscopy can show the presence of discrete clopidogrel nanoparticles.

The particle size of the milled clopidogrel particles can also be measured, in Milli Q Water, using a Horiba LA-910 Particle Sizer (Particular Sciences, Hatton Derbyshire, England). A composition having a D50 particle size of less than 2000 nm meets the criteria of the present invention.

Particle size can be measured initially and after 60 seconds of sonication. Particle sizes that vary significantly following sonication are undesirable, as it is indicative of the presence of clopidogrel aggregates. Such aggregates result in compositions having highly variable particle sizes. Such highly variable particle sizes can result in variable absorption between dosages of a drug, and therefore are undesirable.

It will be apparent to those skilled in the art that various modifications and variations can be made in the methods and compositions of the present inventions without departing from the spirit or scope of the invention. Thus, it is intended that the present invention cover the modification and variations of the invention provided they come within the scope of the appended claims and their equivalents.

What is claimed is:

1. A stable nanoparticulate clopidogrel composition comprising:
(a) particles of clopidogrel or a derivative or a salt thereof having an effective average particle size of less than about 2000 nm; and
(b) at least one surface stabilizer.

2. The composition of claim 1, wherein the nanoparticulate clopidogrel particle is selected from the group consisting of a crystalline phase, an amorphous phase, a semi-crystalline phase, a semi-amorphous phase, and mixtures thereof.

3. The composition of claim 1, wherein the effective average particle size of the nanoparticulate clopidogrel particle is selected from the group consisting of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 100 nm, less than about 75 nm, and less than about 50 nm.

4. The composition of claim 1, wherein the composition is formulated:
(a) for administration selected from the group consisting of oral, pulmonary, rectal, colonic, parenteral, intracisternal, intravaginal, intraperitoneal, ocular, otic, local, buccal, nasal, and topical administration;
(b) into a dosage form selected from the group consisting of liquid dispersions, gels, aerosols, ointments, creams, lyophilized formulations, tablets, capsules;
(c) into a dosage form selected from the group consisting of controlled release formulations, fast melt formulations, delayed release formulations, extended release formulations, pulsatile release formulations, and mixed immediate release and controlled release formulations; or
(d) any combination of (a), (b), and (c).

5. The composition of claim 1, wherein the composition further comprises one or more pharmaceutically acceptable excipients, carriers, or a combination thereof.

6. The composition of claim 1, wherein:
(a) clopidogrel is present in an amount selected from the group consisting of from about 99.5% to about 0.001%, from about 95% to about 0.1%, and from about 90% to about 0.5%, by weight, based on the total combined weight of clopidogrel and at least one surface stabilizer, not including other excipients;
(b) the surface stabilizer is present in an amount selected from the group consisting of about 0.5% to about 99.999% by weight, from about 5.0% to about 99.9% by weight, and from about 10% to about 99.5% by weight, based on the total combined dry weight of clopidogrel and at least one surface stabilizer, not including other excipients; or
(c) a combination thereof.

7. The composition of claim 1, further comprising at least one primary surface stabilizer and at least one secondary surface stabilizer.

8. The composition of claim 1, wherein the surface stabilizer is selected from the group consisting of an anionic surface stabilizer, a cationic surface stabilizer, a non-ionic surface stabilizer, a zwitterionic surface stabilizer, and an ionic surface stabilizer.

9. The composition of claim 1, wherein the surface stabilizer is selected from the group consisting of cetyl pyridinium chloride, gelatin, casein, phosphatides, dextran, glycerol, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, dodecyl trimethyl ammonium bromide, polyoxyethylene stearates, colloidal silicon dioxide, phosphates, sodium dodecylsulfate, carboxymethylcellulose calcium, hydroxypropyl celluloses, hypromellose, carboxymethylcellulose sodium, methylcellulose, hydroxyethylcellulose, hypromellose phthalate, noncrystalline cellulose, magnesium aluminum silicate, triethanolamine, polyvinyl alcohol, polyvinylpyrrolidone, 4-(1,1,3,3-tetramethylbutyl)-phenol polymer with ethylene oxide and formaldehyde, poloxamers; poloxamines, a charged phospholipid, dioctylsulfosuccinate, dialkylesters of sodium sulfosuccinic acid, sodium lauryl sulfate, alkyl aryl polyether sulfonates, mixtures of sucrose stearate and sucrose distearate, p-isononylphenoxypoly-(glycidol), decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; lysozyme, PEG-phospholipid, PEG-cholesterol, PEG-cholesterol derivative, PEG-vitamin A, PEG-vitamin E, lysozyme, random copolymers of vinyl acetate and vinyl pyrrolidone, a cationic polymer, a cationic biopolymer, a cationic polysaccharide, a cationic cellulosic, a cationic alginate, a cationic nonpolymeric compound, a cationic phospholipid, cationic lipids, polymethylmethacrylate trimethylammonium bromide, sulfonium compounds, polyvinylpyrrolidone-2-dimethylaminoethyl methacrylate dimethyl sulfate, hexadecyltrimethyl ammonium bromide, phosphonium compounds, quaternary ammonium compounds, benzyl-di(2-chloroethyl)ethylammonium bromide, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium chloride bromide, C₁₂₋₁₅dimethyl hydroxyethyl ammonium chloride, C₁₂₋₁₅dimethyl hydroxyethyl ammonium chloride bromide, coconut dimethyl hydroxyethyl ammonium chloride, coconut dimethyl hydroxyethyl ammonium bromide, myristyl trimethyl ammonium methyl sulphate, lauryl dimethyl benzyl ammonium chloride, lauryl dimethyl benzyl ammonium bromide, lauryl dimethyl (ethenoxy)₄ ammonium chloride, lauryl dimethyl (ethenoxy)₄ ammonium bromide, N-alkyl (C₁₂₋₁₈)dimethylbenzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts, dialkyl-dimethylammonium salts, lauryl trimethyl ammonium chloride, ethoxylated alkyamidoalkyldialkylammonium salt, an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂ trimethyl ammonium bromides, C₁₅ trimethyl ammonium bromides, C₁₇ trimethyl ammonium bromides, dodecylbenzyl triethyl ammonium chloride, poly-diallyldimethylammonium chloride, dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride, tetrabutylammonium bromide, benzyl trimethylammonium bromide, choline esters, benzalkonium chloride, stearalkonium chloride compounds, cetyl pyridinium bromide, cetyl pyridinium chloride, halide salts of quaternized polyoxyethylalkylamines, alkyl pyridinium salts; amines, amine salts, amine oxides, imide azolinium salts, protonated quaternary acrylamides, methylated quaternary polymers, and cationic guar.

10. The composition of claim 1, wherein the composition does not produce significantly different absorption levels when administered under fed as compared to fasting conditions.

11. The composition of claim 1, wherein administration of the composition to a subject in a fasted state is bioequivalent to administration of the composition to a subject in a fed state.

12. The composition of claim 1, additionally comprising one or more active agents useful for the treatment of pathologies induced by platelet aggregation.

13. The composition of claim 12, wherein the active agent is selected from a group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, and anti-androgens.

14. A stable nanoparticulate clopidogrel composition comprising:
(a) particles of clopidogrel or a derivative or a salt thereof having an effective average particle size of less than about 2000 nm; and
(b) at least one surface stabilizer,
wherein upon administration to a mammal the composition produces therapeutic results at a dosage which is less than that of a non-nanoparticulate dosage form of the same clopidogrel.

15. A clopidogrel composition comprising clopidogrel or a derivative or a salt thereof, wherein the composition has:
(a) a Cₘₐₓ for clopidogrel when assayed in the plasma of a mammalian subject following administration that is greater than the Cₘₐₓ for a non-nanoparticulate formulation of the same clopidogrel, administered at the same dosage;
(b) an AUC for clopidogrel when assayed in the plasma of a mammalian subject following administration that is greater than the AUC for a non-nanoparticulate formulation of the same clopidogrel, administered at the same dosage;
(c) a Tₘₐₓ for clopidogrel when assayed in the plasma of a mammalian subject following administration that is less than the Tₘₐₓ for a non-nanoparticulate formulation of the same clopidogrel, administered at the same dosage; or
(d) any combination of (a), (b), and (c).

16. A method for the preparation of nanoparticulate clopidogrel or a derivative or salt thereof comprising contacting particles of clopidogrel with at least one surface stabilizer for a time and under conditions sufficient to provide a nanoparticulate clopidogrel composition having an effective average particle size of less than about 2000 nm.

17. The method of claim 16, wherein the contacting comprises grinding, wet grinding, homogenization, freezing, template emulsion, precipitation, or a combination thereof.

18. A method for the treatment of pathologies induced by platelet aggregation in a subject comprising administering to a subject a stable nanoparticulate clopidogrel composition comprising:
(a) particles of at least one clopidogrel or a derivative or a salt thereof having an effective average particle size of less than about 2000 nm; and
(b) at least one surface stabilizer.

19. The method of claim 18 wherein the subject is a survivor of a thrombotic event or wherein the subject is at high risk for a thrombotic event.

20. The method of claim 18, wherein the pathology induced by platelet aggregation is a cardiovascular or cerebrovascular disease.

21. The method of claim 18, wherein the treatment is prophylactic.

22. The method of claim 18, wherein the effective average particle size of the nanoparticulate clopidogrel particles is selected from the group consisting of less than about 1900 nm, less than about 1800 nm, less than about 1700 nm, less than about 1600 nm, less than about 1500 nm, less than about 1000 nm, less than about 1400 nm, less than about 1300 nm, less than about 1200 nm, less than about 1100 nm, less than about 900 nm, less than about 800 nm, less than about 700 nm, less than about 600 nn, less than about 500 nm, less than about 400 nm, less than about 300 nm, less than about 250 nm, less than about 200 nm, less than about 100 nm, less than about 75 nm, and less than about 50 nm.
